(19) United States
(12) Patent Application Publication
(10) Pub. No.: US 2006/0194860 A1
(43) Pub. Date: Aug. 31, 2006

(54) Pharmaceutical compositions containing aryl or heteroaryl azolylcarbinol compounds

(75) Inventors: Clemens Gillen, Aachen (DE); Thomas Christoph, Aachen (DE); Klaus Schiene, Duesseldorf (DE); Derek Saunders, Aachen (DE); Hagen-Heinrich Hennies, Simmerath (DE); Carsten Griebel, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: GRUENENTHAL GMBH, AACHEN (DE)

(21) Appl. No.: 11/342,852

(22) Filed: Jan. 31, 2006

Related U.S. Application Data
(63) Continuation of application No. PCT/EP04/08391, filed on Jul. 27, 2004.

(30) Foreign Application Priority Data
Jul. 31, 2003 (DE) 103 35 566.9

(51) Int. Cl. A61K 31/415

(52) U.S. Cl. 514/406

(57) ABSTRACT

Pharmaceutical formulations are provided and comprise compounds corresponding to formula I, 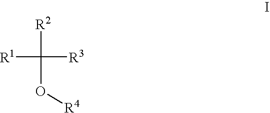
wherein R¹, R², R³ and R⁴ have the meanings given in the description. Related methods of treating or inhibiting pain and other conditions or disorders are also provided.

### CROSS REFERENCE TO RELATED APPLICATIONS

This application is a continuation of International patent application Ser. No. PCT/EP2004/008391, filed Jul. 27, 2004, designating the United States of America and published in German as WO 2005/011684 A1, the entire disclosure of which is incorporated herein by reference. Priority is claimed based on Federal Republic of Germany patent application no. DE 103 35 566.9, filed Jul. 31, 2003.

### FIELD OF THE INVENTION

The invention relates to pharmaceutical compositions containing derivatives of aryl (or heteroaryl) azolyl carbinols and to the use of these derivatives for producing a pharmaceutical composition for the treatment or inhibition of pain, depression, anxiety, urinary incontinence and other syndromes and in related methods of treatment or inhibition.

### BACKGROUND OF THE INVENTION

The treatment of chronic and non-chronic pain is very important in medicine. This is reflected in the large number of publications and research projects. Opioids have accordingly been used for many years as analgesics for the treatment of pain, even though they lead to a number of side effects, for example addiction and dependency, respiratory depression, gastrointestinal obstruction and constipation. Therefore, they can only be given for a prolonged period or in relatively high doses if special precautions such as specific regulations are adhered to (Goodman, Gilman in “The Pharmacological Basis of Therapeutics”, Pergaman Press, New York (1990)).

### SUMMARY OF THE INVENTION

One object of the invention is to provide pharmaceutical compositions, in particular those containing analgesically active substances.

The invention accordingly relates to pharmaceutical compositions containing at least one derivative of an aryl (or heteroaryl) azolyl carbinol according to general formula I 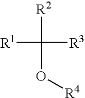
wherein
R¹ is selected from phenyl, unsubstituted or substituted with 1, 2 or 3 substituents selected independently of one another from F, Cl, Br, OCH₃, CH₃ or CF₃;
R² is selected from H or substituted or unsubstituted, saturated or unsaturated, branched or unbranched C₁₋₄ alkyl;
R³ is selected from a heteroaromatic five-membered ring with 1, 2 or 3 nitrogen atoms in the ring, unsubstituted or substituted with 1 or 2 substituents selected independently of one another from F, Cl, Br or CH₃; and
R⁴ is selected from 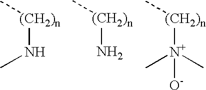
where n=1 or 2;

optionally in the form of their racemates, their pure stereoisomers, in particular enantiomers or diastereomers, or in the form of mixtures of stereoisomers, in particular of enantiomers or diastereomers, in any mixing ratio; in the prepared form or in the form of their acids or bases or in the form of their salts, in particular the physiologically acceptable salts or in the form of their solvates, in particular the hydrates; and optionally additives and/or auxiliaries.

The compounds contained are inhibitors of serotonin uptake, 5HT reuptake, and act as analgesics or else as antidepressants, as also in other indications.

In the context of this invention, alkyl radicals are taken to mean saturated and unsaturated, branched, unbranched and cyclic hydrocarbons which may be unsubstituted or singly or multiply substituted. In this case, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4 or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7 or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. Alkyl, methyl, ethyl, vinyl (ethenyl), propyl, allyl (2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, CHF₂, CF₃ or CH₂OH are preferred.

In this case, in conjunction with alkyl—unless this is not explicitly defined otherwise—the term substituted in the context of this invention is taken to mean the substitution of at least one (optionally also more) hydrogen radical(s) by F, Cl, Br, I, NH₂, SH or OH, wherein “multiply substituted” or “substituted” with multiple substitution is taken to mean that the substitution is made both on different and on the same atoms multiply with the same or different substituents, for example threefold on the same carbon atom as in the case of CF₃ or at different points as in the case of —CH(OH)— CH═CH—CHCl₂. Particularly preferred substituents in this case are F, Cl and OH. With respect to cycloalkyl, the hydrogen radical can also be replaced by OC₁₋₃ alkyl or C₁₋₃ alkyl (singly or multiply substituted or unsubstituted respectively), in particular methyl, ethyl, n-propyl, i-propyl, CF₃, methoxy or ethoxy.

The term (CH₂)₃₋₆ is taken to mean —CH₂—CH₂—CH₂—, —CH₂—CH₂—CH₂—CH₂—, —CH₂—CH₂—CH₂—CH₂—CH₂— and —CH₂—CH₂—CH₂—CH₂—CH₂—CH₂—, (CH₂)₁₋₂ is taken to mean —CH₂— or —CH₂—CH₂—, (CH₂)₁₋₄ is taken to mean —CH₂—, —CH₂—CH₂—, —CH₂—CH₂—CH₂— and —CH₂—CH₂—CH₂—CH₂, (CH₂)₄₋₅ is taken to mean —CH₂—CH₂—CH₂—CH₂— and —CH₂—CH₂—CH₂—CH₂—CH₂—, etc.

An aryl radical is taken to mean ring systems with at least one aromatic ring but without heteroatoms in even only one of the rings. Examples are phenyl, naphthyl, fluoranthenyl, fluorenyl, tetralinyl or indanyl, in particular 9H fluorenyl or anthracenyl radicals which may be unsubstituted or singly or multiply substituted.

A heteroaryl radical is taken to mean heterocyclic ring systems with at least one unsaturated ring which contain one or more heteroatoms from the group comprising nitrogen, oxygen and/or sulphur and which can also be singly or multiply substituted. Examples from the group of heteroaryls include furan, benzofuran, thiophene, benzothiophene, pyrrole, pyridine, pyrimidine, pyrazine, quinoline, isoquinoline, phthalazine, benzo[1,2,5]thiadiazole, benzothiazole, indole, benzotriazole, benzodioxolan, benzodioxan, carbazole, indole and quinazoline.

In this case, in conjunction with aryl and heteroaryl, substituted is taken to mean the substitution of the aryl or heteroaryl by R²², OR²² a halogen, preferably F and/or Cl, a CF₃, a CN, a NO₂, a NR²³R²⁴, a C₁₋₆-alkyl (saturated), a C₁₋₆-alkoxy, a C₃₋₈-cycloalkoxy, a C₃₋₈-cycloalkyl or a C₂₋₆-alkylene—unless otherwise defined.

In this case the radical R²² represents H, a C₁₋₁₀ alkyl, preferably a C₁₋₆ alkyl, an aryl or heteroaryl radical or an aryl or heteroaryl radical bound by C₁₋₃ alkyl, saturated or unsaturated, or by a C₁₋₃ alkylene group, wherein these aryl or heteroaryl radicals must not themselves be substituted by aryl or heteroaryl radicals,
the radicals R²³ and R²⁴, which are the same or different, represent H, a C₁₋₁₀ alkyl, preferably a C₁₋₆ alkyl radical, an aryl radical, a heteroaryl radical or an aryl or heteroaryl radical bound by C₁₋₃ alkyl, saturated or unsaturated, or by a C₁₋₃ alkylene group, wherein these aryl or heteroaryl radicals must not themselves be substituted by aryl or heteroaryl radicals,
or the radicals R²³ and R²⁴ together represent CH₂CH₂OCH₂CH₂, CH₂CH₂NR²⁵CH₂CH₂ or (CH₂)₃₋₆, and
the radical R²⁵ represents H, a C₁₋₁₀ alkyl, preferably a C₁₋₆ alkyl, an aryl radical, a heteroaryl radical or an aryl or heteroaryl radical bound by saturated or unsaturated C₁₋₃ alkyl or a C₁₋₃ alkylene-group-bound aryl or heteroaryl radical, wherein these aryl or heteroaryl radicals must not themselves be substituted by aryl or heteroaryl radicals.

The term salt is taken to mean any form of the active ingredient according to the invention in which it assumes or is charged with an ionic form and is coupled to a counter ion (a cation or anion) or is in solution. This also includes complexes of the active ingredient with other molecules and ions, in particular complexes complexed by ionic interactions. In particular, it includes (and this is also a preferred embodiment of this invention) physiologically acceptable salts, in particular physiologically acceptable salts with cations or bases and physiologically acceptable salts with anions or acids or also a salt formed with a physiologically acceptable acid or a physiologically acceptable cation.

The term physiologically acceptable salt with anions or acids is taken to mean, in the context of this invention, salts of at least one of the compounds according to the invention—usually protonated, for example on nitrogen—as cation with at least one anion which are physiologically acceptable—in particular when applied to humans and/or mammals. In the context of this invention this is taken to mean, in particular, the salt formed with a physiologically acceptable acid, namely salts of the respective active ingredient with inorganic or organic acids which are physiologically acceptable—in particular when applied to humans and/or mammals. Examples of physiologically acceptable salts of specific acids are salts of: hydrochloric acid, hydrobromic acid, sulphuric acid, methane sulphonic acid, formic acid, acetic acid, oxalic acid, succinic acid, malic acid, tartaric acid, mandelic acid, fumaric acid, lactic acid, citric acid, glutamic acid, 1,1-dioxo-1,2,dihydro-1b6-benzo[d]isothiazol-3-one (saccharic acid), monomethyl sebacic acid, 5-oxo-proline, hexane-1-sulphonic acid, nicotinic acid, 2-, 3- or 4-aminobenzoic acid, 2,4,6-trimethyl-benzoic acid, a-lipoic acid, acetyl glycine, acetyl salicylic acid, hippuric acid and/or aspartic acid. The hydrochloride salt is particularly preferred.

The term salt formed with a physiologically acceptable acid in the context of this invention is taken to mean salts of the respective active ingredient with inorganic or organic acids which are physiologically acceptable—in particular when applied to humans and/or mammals. The hydrochloride is particularly preferred. Examples of physiologically acceptable acids include: hydrochloric acid, hydrobromic acid, sulphuric acid, methane sulphonic acid, formic acid, acetic acid, oxalic acid, succinic acid, tartaric acid, mandelic acid, fumaric acid, lactic acid, citric acid, glutamic acid, 1,1-dioxo-1,2-dihydro-1λ⁶-benzo[d]isothiazol-3-one (saccharic acid), monomethyl sebacic acid, 5-oxo-proline, hexane-1-sulphonic acid, nicotinic acid, 2-, 3- or 4-aminobenzoic acid, 2,4,6-trimethyl-benzoic acid, α-lipoic acid, acetyl glycine, acetyl salicylic acid, hippuric acid and/or aspartic acid.

The term physiologically acceptable salt with cations or bases is taken to mean, in the context of this invention, salts of at least one of the compounds according to the invention—usually a (deprotonated) acid—as anion with at least one, preferably inorganic, cation which are physiologically acceptable, in particular when applied to humans and/or mammals. The salts of the alkali and alkaline earth metals, but also with NH₄⁺ are particularly preferred, in particular however (mono) or (di) sodium, (mono) or (di) potassium, magnesium or calcium salts.

The term salt formed with a physiologically acceptable cation in the context of this invention is taken to mean salts of at least one of the respective compounds as anion with at least one inorganic cation which is physiologically acceptable—in particular when applied to humans and/or mammals. The salts of the alkali and alkaline earth metals, but also NH₄⁺ are particularly preferred, in particular however (mono) or (di) sodium, (mono) or (di) potassium, magnesium or calcium salts.

Suitable additives and/or auxiliaries in the context of this invention include any substances known to a person skilled in the art from the prior art, for achieving galenical formulations. Auxiliaries may be, for example water, ethanol, 2-propanol, glycerine, ethylene glycol, propylene glycol, polyethylene glycol, polypropylene glycol, glucose, fructose, lactose, saccharose, dextrose, molasses, starch, modified starch, gelatine, sorbitol, inositol, mannitol, microcrystalline cellulose, methylcellulose, carboxymethylcellulose, cellulose acetate, shellac, cetyl alcohol, polyvinyl pyrrolidone, paraffins, waxes, natural and synthetic rubbers, acacia gum, alginates, dextran, saturated and unsaturated fatty acids, stearic acid, magnesium stearate, zinc stearate, glyceryl stearate, sodium lauryl sulphate, edible oils, sesame oil, coconut oil, groundnut oil, soya bean oil, lecithin, sodium lactate, polyoxyethylene and propylene fatty acid esters, sorbitan fatty acid ester, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulphate, zinc sulphate, calcium sulphate, potash, calcium phosphate, dicalcium phosphate, potassium bromide, potassium iodide, talcum, kaolin, pectin, crosprovidone, agar and bentonite.

The choice of these auxiliaries and the quantities thereof to be used depend on whether the pharmaceutical preparation is to be applied orally, intravenously, intraperitoneally, intradermally, intramuscularly, intranasally, buccally or topically. Preparations in the form of tablets, chewable tablets, dragees, capsules, granules, drops, juices or syrups are suitable for oral application, solutions, suspensions, easily reconstitutable dry preparations and sprays are suitable for parenteral, topical and inhalative application. Suppositories for application in the rectum are a further possibility.

The active ingredient may be released from certain forms of preparation after a delay. Examples include retarding forms of tablet but, in particular, also the application of the active ingredient in a deposit, in dissolved form, in a substrate film or in a plaster, optionally with the addition of agents to promote skin penetration, as suitable examples for suitable percutaneous forms of application as well as particles or implants which release after a delay.

Examples of auxiliaries and additives for oral forms of application include disintegrating agents, lubricants, binders, fillers, mould release agents, optionally solvents, flavourings, sugar, in particular excipients, diluents, dyes, antioxidants, etc. Waxes and fatty acid esters, inter alia, may be used for suppositories and carriers, preservatives, suspension aids, etc. for parentral forms of application.

A particularly preferred form of the pharmaceutical composition according to the invention exists when the pharmaceutical composition displays delayed release, and is preferably in the form of a retard formulation, in particular is in the form of a particle or implant having delayed release, preferably plastic implant or particle, the plastic preferably being selected from polylactide or a polylactide/polyglycolide copolymer or is a transdermal therapeutic system in the form of a plaster.

The medicaments and pharmaceutical compositions according to the invention may be produced using means, devices, methods and processes well known in the prior art of pharmaceutical formulation and described, for example, in Remington's Pharmaceutical Sciences, A. R. Gennaro, 17 Ed., Mack Publishing Company, Easton, Pa. (1985), in particular in Part 8, Chapters 76 to 93. However, other methods of production are also conceivable and known, in particular for modern forms of pharmaceutical composition.

For preferred pharmaceutical compositions according to the invention, R² is selected from H, CH₃, C₂H₅, i-propyl, n-propyl, n-butyl, i-butyl, sec.-butyl and tert. butyl.

It is greatly preferred if, in the pharmaceutical composition according to the invention, the derivative of an aryl (or heteroaryl) azolyl carbinol according to general formula I is selected from: 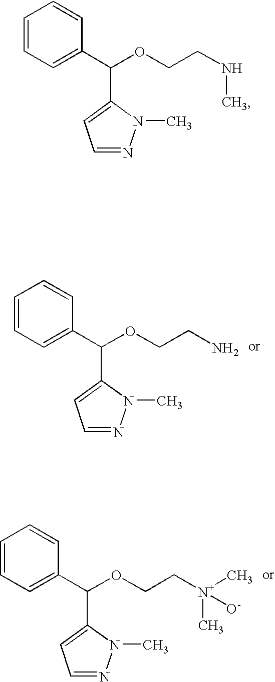
5-{α- [2-(methylamino)ethoxy]benzyl}-1-methyl-1H-pyrazole,
5-[α-(2-aminoethoxy)benzyl]-1-methyl-1H-pyrazole, or
5-{α-[2-(dimethylamino-N-oxide)ethoxy]benzyl}-1-methyl-1H-pyrazole,
optionally in the form of their racemates, their pure stereoisomers, in particular enantiomers or diastereomers, or in the form of mixtures of stereoisomers, in particular of enantiomers or diastereomers, in any mixing ratio; in the prepared form or in the form of their acids or bases or in the form of their salts, in particular the physiologically acceptable salts or in the form of their solvates, in particular the hydrates;
   in particular
(±)-5-{α-[2-(methylamino)ethoxy]benzyl}-1-methyl-1H-pyrazole,
(+)-5-{α-[2-(methylamino)ethoxy]benzyl}-1-methyl-1H-pyrazole,
(−)-5-{α-[2-(methylamino)ethoxy]benzyl}-1-methyl-1H-pyrazole,
(±)-5-[α-(2-aminoethoxy)benzyl]-1-methyl-1H-pyrazole,
(+)-5-[α-(2-aminoethoxy)benzyl]-1-methyl-1H-pyrazole,
(−)-5-[α-(2-aminoethoxy)benzyl]-1-methyl-1H-pyrazole,
(±)-5-{α-[2-(dimethylamino-N-oxide)ethoxy]benzyl}-1-methyl-1H-pyrazole,
(+)-5-{α-[2-(dimethylamino-N-oxid)ethoxy]benzyl}-1-methyl-1H-pyrazole,
or (−)-5-{α-[2-(dimethylamino-N-oxide)ethoxy]benzyl}-1-methyl-1H-pyrazole,
   preferably
5-{α-[2-(methylamino)ethoxy]benzyl}-1-methyl-1H-pyrazole,
(±)-5-{α-[2-(methylamino)ethoxy]benzyl}-1-methyl-1H-pyrazole,
(+)-5-{α-[2-(methylamino)ethoxy]benzyl}-1-methyl-1H-pyrazole or
(−)-5-{α-[2-(methylamino)ethoxy]benzyl}-1-methyl-1H-pyrazole.

These last-mentioned compounds are metabolites of cizolirtine. Cizolirtine is a compound, (2-[phenyl(1-methyl-1H-pyrazol-5-yl)methoxy]-N,N-dimethylethan-amine, known to a person skilled in the art and described in European patent EP 289380 B1, which may also be described as 5-[alpha-(2-dimethylaminoethoxy)-benzyl]-1-methyl-1H-pyrazole or 5-{[N,N-dimethylaminoethoxy)phenyl]methyl}-1-methyl-1H-pyrazole) of formula II:

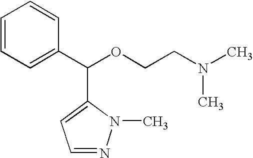

The production and use of cizolirtine as an analgesic is described therein—but also in various publications.

Two enantiomers of the compound were accordingly synthesised and their properties as an analgesic was investigated [J. A. Hueso, J. Berrocal, B. Gutierrez, A. J. Farre and J. Frigola, Biorg. Med. Chem. Lett. 1993, 3, 269]. It was found that the dextrorotatory enantiomer was the more active.

The metabolites of cizolirtine are described by Martinez et al. 1999, Xenobiotica, 29(8); 859-871 and in the publication by Gómez-Gomar et al., 2002, Journal of Chromatography A, 950, 252-270.

The derivatives of aryl (or heteroaryl) azolyl carbinols contained in the pharmaceutical compositions according to the invention may be produced by initially producing the corresponding compounds in which R⁴ represents —(CH₂)ₙ—N—(CH₃)₂. These dimethylamino compounds are then reacted with a microsome fraction under suitable physiological conditions and the metabolites in the compounds contained in the pharmaceutical compositions according to the invention are separated and purified by chromatography. This can be carried out simultaneously with the procedure described by Martinez et al. 1999, Xenobiotica, 29(8); 859-871 for cizolirtine as the amino compound used. In particular, this procedure therefore obviously applies to the preferably contained metabolites of cizolirtine.

The derivatives of aryl (or heteroaryl) azolyl carbinols contained in the pharmaceutical compositions according to the invention are high-affinity inhibitors of serotonin uptake, of the so-called 5HT reuptake. Accordingly, they are eminently suitable for the treatment of pain, depression, anxiety and urinary incontinence.

Therefore, the invention further relates to the use of a derivative of an aryl (or heteroaryl) azolyl carbinol according to general formula I 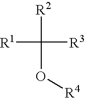
wherein
R¹ is selected from phenyl, unsubstituted or substituted with 1, 2 or 3 substituents selected independently of one another from F, Cl, Br, OCH₃, CH₃ or CF₃;
R² is selected from H or substituted or unsubstituted, saturated or unsaturated, branched or unbranched C₁₋₄ alkyl;
R³ is selected from a heteroaromatic five-membered ring with 1, 2 or 3 nitrogen atoms in the ring, unsubstituted or substituted with 1 or 2 substituents selected independently of one another from F, Cl, Br or CH₃; and
R⁴ is selected from 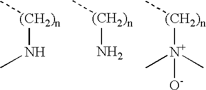
where n=1 or 2;
optionally in the form of its racemates, its pure stereoisomers, in particular enantiomers or diastereomers, or in the form of mixtures of stereoisomers, in particular of enantiomers or diastereomers, in any mixing ratio; in the prepared form or in the form of its acids or bases or in the form of its salts, in particular the physiologically acceptable salts or in the form of its solvates, in particular the hydrates; for producing a pharmaceutical composition for the treatment of pain, in particular neuropathic, chronic, visceral, inflammatory or cancer pain; depression, anxiety, urinary discomfort, increased frequency of miction and/or urinary incontinence.

Urinary incontinence is involuntary urination. This occurs in an uncontrolled manner when the pressure within the urinary bladder exceeds the pressure required to close the urinary duct. The causes may be, on the one hand, increased internal bladder pressure (for example due to detrusor instability) with the resultant stress incontinence and, on the other hand, reduce sphincter pressure (for example after childbirth or surgical operations) with the resultant stress incontinence. The detrusor is the coarsely bundled multi-layered bladder wall musculature, of which the contraction leads to emptying of the bladder, and the sphincter is the closing muscle complex of the urethra. Mixed forms of these types of incontinence and the so-called overflow incontinence (for example in the case of benign prostate hyperplasia) or reflex incontinence (for example after damage to the spinal cord) occur. Further details can be found in Chutka, D. S. and Takahashi, P. Y., 1998, Drugs 560: 587-595.

Urinary discomfort is the state of increased bladder muscle tension which leads to emptying of the bladder (miction) as the bladder nears its capacity (or its capacity is exceeded). The syndrome of stress incontinence includes 1. increased urinary discomfort; 2. increased frequency of miction; and 3. involuntary urinary incontinence as such. Causes may be inter alia inflammation of the urinary bladder and neurogenic bladder disorders as well tuberculosis of the bladder. However, the causes have not yet all been explained. The overactive bladder is a further syndrome which belongs here.

According to findings in research into pharmaceutical compositions, the inhibition of serotonin uptake is also a clear sign of the effectiveness in other indications.

Therefore, the invention further relates to the use of a derivative of an aryl (or heteroaryl) azolyl carbinol according to general formula I

wherein

R¹ is selected from phenyl, unsubstituted or substituted with 1, 2 or 3 substituents selected independently of one another from F, Cl, Br, OCH₃, CH₃ or CF₃;
R² is selected from H or substituted or unsubstituted, saturated or unsaturated, branched or unbranched C₁₋₄ alkyl;
R³ is selected from a heteroaromatic five-membered ring with 1, 2 or 3 nitrogen atoms in the ring, unsubstituted or substituted with 1 or 2 substituents selected independently of one another from F, Cl, Br or CH₃; and
R⁴ is selected from 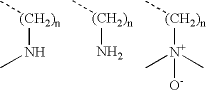
where n=1 or 2;
optionally in the form of its racemates, its pure stereoisomers, in particular enantiomers or diastereomers, or in the form of mixtures of stereoisomers, in particular of enantiomers or diastereomers, in any mixing ratio; in the prepared form or in the form of its acids or bases or in the form of its salts, in particular the physiologically acceptable salts or in the form of its solvates, in particular the hydrates; for producing a pharmaceutical composition for the treatment of schizophrenia, psychoses, phobias, manic depressive psychoses, withdrawal from nicotine or drugs, drug dependency, cognitive dysfunction, Parkinson's disease, cardiac-circulatory diseases, ischaemia, strokes, ADHD (attention deficit hyperactivity disorder), eating disorders, premenstrual syndrome (PMS), OCD (obsessive-compulsive disorder), PTSD (posttraumatic stress disorder) and/or motor problems.

For all the aforementioned uses according to the invention, it is desirable if R² is selected from H, CH₃, C₂H₅, i-propyl, n-propyl, n-butyl, i-butyl, sec.-butyl and tert. butyl.

For all the aforementioned uses according to the invention, it is quite particularly preferred if the used derivative of an aryl (or heteroaryl) azolyl carbinol according to general formula I is selected from: 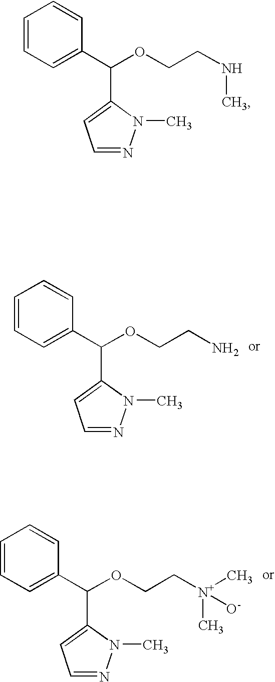
5-{α-[2-(methylamino)ethoxy]benzyl}-1-methyl-1H-pyrazole,
5-[α-(2-aminoethoxy)benzyl]-1-methyl-1H-pyrazole, or
5-{α-[2-(dimethylamino-N-oxide)ethoxy]benzyl}-1-methyl-1H-pyrazole,
optionally in the form of their racemates, their pure stereoisomers, in particular enantiomers or diastereomers, or in the form of mixtures of stereoisomers, in particular of enantiomers or diastereomers, in any mixing ratio; in the prepared form or in the form of their acids or bases or in the form of their salts, in particular the physiologically acceptable salts or in the form of their solvates, in particular the hydrates;
   in particular
(±)-5-{α-[2-(methylamino)ethoxy]benzyl}-1-methyl-1H-pyrazole,
(+)-5-{α-[2-(methylamino)ethoxy]benzyl}-1-methyl-1H-pyrazole,
(−)-5-{α-[2-(methylamino)ethoxy]benzyl}-1-methyl-1H-pyrazole,
(±)-5-[α-(2-aminoethoxy)benzyl]-1-methyl-1H-pyrazole,
(+)-5-[α-(2-aminoethoxy)benzyl]-1-methyl-1H-pyrazole,
(−)-5-[α-(2-aminoethoxy)benzyl]-1-methyl-1H-pyrazole,
(±)-5-{α-[2-(dimethylamino-N-oxide)ethoxy]benzyl}-1-methyl-1H-pyrazole,
(+)-5-{α-[2-(dimethylamino-N-oxid)ethoxy]benzyl}-1-methyl-1H-pyrazole, or
(−)-5-{α-[2-(dimethylamino-N-oxide)ethoxy]benzyl}-1-methyl-1H-pyrazole,
   preferably
5-{α-[2-(methylamino)ethoxy]benzyl}-1-methyl-1H-pyrazole,
(±)-5-{α-[2-(methylamino)ethoxy]benzyl}-1-methyl-1H-pyrazole,
(+)-5-{α-[2-(methylamino)ethoxy]benzyl}-1-methyl-1H-pyrazole or
(−)-5-{α-[2-(methylamino)ethoxy]benzyl}-1-methyl-1H-pyrazole.

The invention further relates to a method for the treatment of pain, in particular neuropathic, chronic, visceral, inflammatory or cancer pain; depression, anxiety, increased urinary discomfort, increased frequency of miction and/or urinary incontinence, in which at least one pharmaceutical composition according to the invention is used.

The invention also relates to a method for the treatment of schizophrenia, psychoses, phobias, manic depressive psychoses, withdrawal from nicotine or drugs, drug dependency, cognitive dysfunction, Parkinson's disease, cardiac-circulatory diseases, ischaemia, strokes, ADHD (attention deficit hyperactivity disorder), eating disorders, premenstrual syndrome (PMS), OCD (obsessive-compulsive disorder), PTSD (posttraumatic stress disorder) and/or mobility problems, in which at least one pharmaceutical composition according to the invention is used.

Certain embodiments of the present invention may be further understood by reference to the following specific examples. These examples and the terminology used herein are for the purpose of describing particular embodiments only and are not intended to be limiting.

### EXAMPLES

### Example 1

### Functional Measurement of the 5-HT-Uptake or the Inhibition of Serotonin Uptake

The uptake of tritium-marked 5-HT (serotonin) as a function of the concentration of the added substance 5-{α-[2-(methylamino)ethoxy]benzyl}-1-methyl-1H-pyrazole in a system with neurological tissue under physiological conditions. The result of the investigations is shown in FIG. 1. A Kᵢ of 0.53±0.10 μm in the inhibition of serotonin uptake was found for the substance 5-{α-[2-(methylamino)ethoxy]benzyl}-1-methyl-1H-pyrazole from three independent tests.

This demonstrates that this substance is a high-affinity inhibitor of the 5-HT reuptake and is therefore effective in pain, depression and other indications.

The foregoing description and example have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A pharmaceutical composition comprising a compound corresponding to formula I 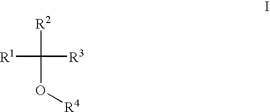
wherein
R¹ is selected from phenyl which is unsubstituted or substituted with 1, 2 or 3 substituents selected independently of one another from F, Cl, Br, OCH₃, CH₃ or CF₃;
R² is H or is C₁₋₄ alkyl which is substituted or unsubstituted, saturated or unsaturated and branched or unbranched;
R³ is selected from a heteroaromatic five-membered ring with 1, 2 or 3 nitrogen atoms in the ring which is unsubstituted or substituted with 1 or 2 substituents selected independently of one another from F, Cl, Br or CH₃; and
R⁴ is selected from 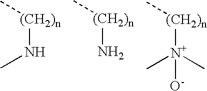
where n=1 or 2,
or a physiologically acceptable salt thereof and
an auxiliary agent.

2. The compound of claim 1, wherein said compound is present in the form of a base.

3. The compound of claim 1, wherein said compound is present in the form of an acid.

4. The compound of claim 1, wherein said compound is present in the form of a pure enantiomer or pure diastereoisomer.

5. The compound of claim 1, wherein said compound is present in the form of a mixture of stereoisomers.

6. The compound of claim 1, wherein said compound is present in the form of a racemic mixture.

7. The compound of claim 1, wherein said compound is present in the form of a solvate.

7. The compound of claim 1, wherein said compound is present in the form of a hydrate.

8. A pharmaceutical composition according to claim 1, wherein R² is selected from H, CH₃, C₂H₅, i-propyl, n-propyl, n-butyl, i-butyl, sec.-butyl and tert. butyl.

9. A pharmaceutical composition according to claim 1, wherein the compound corresponding to formula I is selected from the group consisting of: 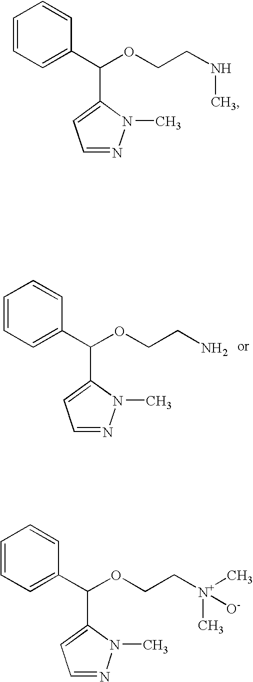
5-{α-[2-(methylamino)ethoxy]benzyl}-1-methyl-1H-pyrazole,
5-[α-(2-aminoethoxy)benzyl]-1-methyl-1H-pyrazole, and
5-{α-[2-(dimethylamino-N-oxide)ethoxy]benzyl}-1-methyl-1H-pyrazole.

10. A pharmaceutical composition according to claim 1, wherein the compound corresponding to formula I is selected from the group consisting of:
(±)-5-{α-[2-(methylamino)ethoxy]benzyl}-1-methyl-1H-pyrazole,
(+)-5-{α-[2-(methylamino)ethoxy]benzyl}-1-methyl-1H-pyrazole,
(−)-5-{α-[2-(methylamino)ethoxy]benzyl}-1-methyl-1H-pyrazole,
(±)-5-[α-(2-aminoethoxy)benzyl]-1-methyl-1H-pyrazole,
(+)-5-[α-(2-aminoethoxy)benzyl]-1-methyl-1H-pyrazole,
(−)-5-[α-(2-aminoethoxy)benzyl]-1-methyl-1H-pyrazole,
(±)-5-{α-[2-(dimethylamino-N-oxide)ethoxy]benzyl}-1-methyl-1H-pyrazole,
(+)-5-{α-[2-(dimethylamino-N-oxid)ethoxy]benzyl}-1-methyl-1H-pyrazole, and
(−)-5-{α-[2-(dimethylamino-N-oxide)ethoxy]benzyl}-1-methyl-1H-pyrazole.

11. A pharmaceutical composition according to claim 1, wherein the compound corresponding to formula I is selected from the group consisting of:
5-{α-[2-(methylamino)ethoxy]benzyl}-1-methyl-1H-pyrazole,
(±)-5-{α-[2-(methylamino)ethoxy]benzyl}-1-methyl-1H-pyrazole,
(+)-5-{α-[2-(methylamino)ethoxy]benzyl}-1-methyl-1H-pyrazole and
(−)-5-{α-[2-(methylamino)ethoxy]benzyl}-1-methyl-1H-pyrazole.

12. A method of treating or inhibiting a condition selected from the group consisting of: pain, depression, anxiety, increased urinary discomfort, increased frequency of miction and urinary incontinence said method comprising administering to a subject in need thereof a pharmaceutically effective amount of a compound corresponding to formula I of claim 1.

13. The method of claim 12, wherein said pain is neuropathic, chronic, inflammatory or cancer pain.

14. A method of treating or inhibiting a condition selected from the group consisting of: schizophrenia, psychoses, phobias, manic depressive psychoses, withdrawal from nicotine or drugs, drug dependency, cognitive dysfunction, Parkinson's disease, cardiac-circulatory diseases, ischaemia, strokes, attention deficit hyperactivity disorder, eating disorders, premenstrual syndrome, obsessive-compulsive disorder, posttraumatic stress disorder and mobility problems, said method comprising administering to a subject in need thereof a pharmaceutically effective amount of a compound corresponding to formula I of claim 1.
